(19) United States
(12) Patent Application Publication
(10) Pub. No.: US 2023/0129946 A1
(43) Pub. Date: Apr. 27, 2023

(54) SMART MATERIAL SAMPLE ANALYSIS

(71) Applicant: Principia Life, LLC, Bothell, WA (US)

(72) Inventor: Darren KRESS, Bothell, WA (US)

(21) Appl. No.: 17/961,482

(22) Filed: Oct. 6, 2022

Related U.S. Application Data
(60) Provisional application No. 63/271,393, filed on Oct. 25, 2021.

(51) Int. Cl. G16H 30/40; G16H 50/20; G06T 7/00; G06F 21/62

(52) U.S. Cl. CPC G16H 30/40; G16H 50/20; G06T 7/0012; G06F 21/6254; G06T 2207/10056; G06T 2207/10064; G06T 2207/20081; G06T 2207/30024

(57) ABSTRACT

Described herein are techniques for determining and executing data collection instructions for a material sample collection device. Such techniques may comprise receiving, at the sample collection device, a material sample, obtaining at least one initial image of the material sample, determining, based on the at least one initial image, a category for the material sample, retrieving, based on the determined category for the material sample, a set of data collection instructions specific to the determined category, collecting a set of images in accordance with the retrieved set of data collection instructions, and transmitting the set of images to a service provider platform.

### CROSS-REFERENCE TO RELATED PATENT APPLICATION

This application claims priority to U.S. Provisional Patent Application No. 63/271,393, filed on Oct. 25, 2021, entitled “Smart Material Sample Collection Device,” which is hereby incorporated by reference in its entirety.

### BACKGROUND

As a result of the pandemic, medical facilities have recently become increasingly crowded and overutilized. However, such crowding can result in increased spreading of a pandemic infection as uninfected individuals can become increasingly exposed to infected individuals in these crowded spaces. Accordingly, there is a need for remote diagnosis tools that can be used by unsophisticated users, such as those who have no medical training.

### SUMMARY

Techniques are described herein for determining and executing data collection instructions on a material sample collection device. Such techniques comprise determining, using an initial image of the material sample, a category or type of that sample. In some embodiments, the initial image is provided to a trained machine learning model included on the sample collection device. The machine learning model identifies a likely type of the sample. Based on the identified type of sample, the sample collection device may retrieve a set of data collection instructions. A set of data analysis instructions may also be retrieved. The sample collection device may then proceed to collect images in accordance with the retrieved set of data collection instructions. The collected images may then be analyzed to determine one or more conditions affecting the collected sample. In some embodiments, this comprises providing the collected images to a remote server to be processed. In some embodiments, this comprises processing the collected images locally with retrieved data analysis instructions so that a diagnosis can be made.

The foregoing, together with other features and embodiments, will become more apparent upon referring to the following specification, claims, and accompanying drawings.

### BRIEF DESCRIPTION OF THE DRAWINGS

The detailed description is described with reference to the accompanying figures, in which the left-most digit(s) of a reference number identifies the figure in which the reference number first appears. The use of the same reference numbers in different figures indicates similar or identical items.

FIG. 1 depicts an illustrative system that may be implemented in accordance with various embodiments of the disclosure;

FIG. 2 depicts an example of a second illustrative system that may be implemented in accordance with various embodiments of the disclosure;

FIG. 3 depicts a first example of a collection cartridge having a single testing compartment that may be used along with a sample collection device in accordance with at least some embodiments;

FIG. 4 depicts a second example of a collection cartridge having multiple testing compartments that may be used along with a sample collection device in accordance with at least some embodiments;

FIG. 5 depicts a third example of a collection cartridge that may be used along with a sample collection device in accordance with at least some embodiments;

FIG. 6 depicts a fourth example of a collection cartridge that may be used along with a sample collection device in accordance with at least some embodiments;

FIG. 7 depicts a block diagram illustrating various features of a sample collection device in accordance with at least some embodiments;

FIG. 8 depicts a flow diagram illustrating an exemplary process for determining a sample collection strategy and collecting images in accordance with embodiments; and

FIG. 9 illustrates an exemplary overall training process of training a machine learning model to optimize collection of material sample data in accordance with aspects of the disclosed subject matter.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Described herein are techniques for determining and executing data collection instructions on a material sample collection device. A material sample may comprise any suitable sample of a material to be tested. In some cases, the material sample may comprise a biological sample or a non-biological sample. The biological sample may include bodily fluids, tissue, bodily excretions, and/or other biological specimens collected from a user or an environment surrounding the user. The non-biological samples may include water samples, dirt samples, and/or samples of other non-biological particulates that are collected from the user or the environment that surround the user. In some embodiments, an initial image of the material sample may be used to determine a category or type of that sample. In some embodiments, the initial image is provided to a trained machine learning model included on the sample collection device. The machine learning model identifies a likely type of the sample. Based on the identified type of sample, the sample collection device may retrieve a set of data collection instructions. The sample collection device may then proceed to collect images in accordance with the retrieved set of data collection instructions. The collected images may then be processed on the material sample collection device or transmitted to a remote server to be processed so that a diagnosis can be made.

Embodiments of the disclosure provide several advantages over conventional systems. For example, embodiments of the system described herein enable users to receive accurate results from a portable sample collection device regardless of that user's level of sophistication or training. In cases for which diagnosis is to rely upon images captured and provided by a user without medical training, such diagnosis can become skewed or inaccurate if low quality or suboptimal images are collected. Embodiments as described herein enable a user to capture and provide more optimal images for testing, regardless of that user's training.

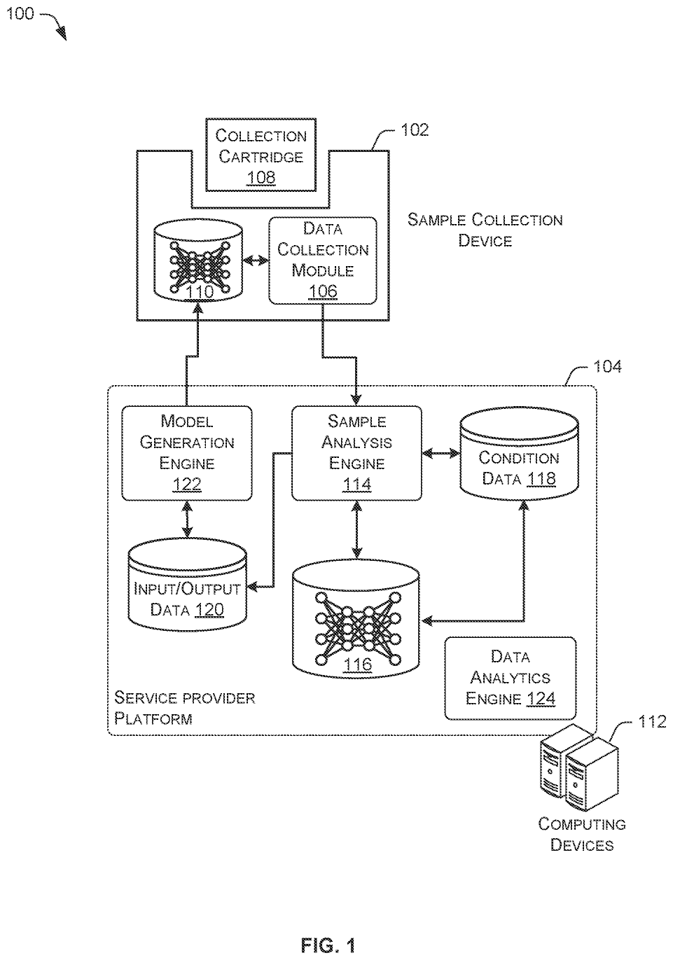

FIG. 1 depicts an illustrative system 100 that may be implemented in accordance with various embodiments of the disclosure. As will be appreciated, although a single type of environment is used for purposes of explanation, different environments may be used, as appropriate, to implement various embodiments. The illustrative system 100 includes a sample collection device 102 that is operable by a user to send and receive data over a network to a service provider platform 104. The sample collection device 102 may have installed upon it a data collection module 106 that performs at least a portion of the functionality described herein and that facilitates collection of material samples by the sample collection device 102. In some embodiments, the sample collection device 102 may be configured to receive a collection cartridge 108 which may be a separate unit that provides for testing of a material sample.

In some embodiments, a collection cartridge 108 may be specific to a particular type of material sample. For example, each collection cartridge 108 may be configured to receive and test a particular type of material sample. In this example, a collection cartridge 108 may include a machine-readable code that indicates the particular type of material specific to the collection cartridge 108.

The sample collection device 102 can include any suitable electronic device operable to collect sample information and to convey that sample information over an appropriate network. In some embodiments, such sample collection devices include electronic devices that are capable of independent communication over a network (e.g., a wireless cellular network). In other embodiments, such sample collection devices include an electronic device that can be coupled to, or otherwise paired with, devices that are capable of network communication, such as personal computers, cell phones, handheld messaging devices, laptop computers, set-top boxes, personal data assistants, electronic book readers and the like. In some embodiments, the sample collection device 102 may include a motor or other mechanical means which may be used to spin, vibrate, or rock the sample collection cartridge 108 in order to mix the material sample and a diluent.

In some embodiments, the sample collection device 102 may be provisioned with at least one machine learning model 110 that has been trained to correlate detected sample information with a number of data collection information/requirements. For example, a trained machine learning model 110 may be configured to, upon being provided an initial set of images for a material sample as input, identify a type of the material sample being analyzed as well as a data collection strategy for that material sample. In some embodiments, identifying a type of the material sample may comprise determining a likely composition/category of the material sample based on an initial set of images (e.g., one or more images of the material sample at specified levels of magnification, etc.) collected by the sample collection device 102. In some embodiments, identifying a type of the material sample may comprise reading an identifier off a machine-readable code included on the collection cartridge 108.

Once a likely composition/category of the material sample has been determined, the data collection module 106 may be configured to execute a set of data collection instructions associated with the identified composition/category. In executing such data collection instructions, the data collection module 106 may be configured to obtain a number of images of the material sample using specified settings (e.g., resolution, level of magnification, etc.). For example, upon determining that the material sample is most likely blood, the data collection module 106 may be configured to cause an image collection device (e.g., a camera) included within the sample collection device 102 to obtain images of the material sample at a range of specified magnifications.

The sample collection device 102 and the service provider platform 104 can include any computing device (e.g., computing device 112) configured to perform at least a portion of the operations described herein. The computing device 112 may be equipped with a communication interface, a user interface, one or more processors, and memory. The communication interface may include wireless and/or wired communication components that enable the computing devices to transmit or receive data via a network, such as the Internet. The user interface may enable a user to provide inputs and receive outputs from the computing devices. The user interface may include a data output device (e.g., visual display, audio speakers), and one or more data input devices. The data input devices may include, but are not limited to, combinations of one or more of keypads, keyboards, mouse devices, touch screens, microphones, speech recognition packages, and any other suitable devices or other electronic/software selection methods. Each of the processors may be a single-core processor or a multi-core processor. Memory may be implemented using computer-readable media, such as computer storage media. Computer-readable media includes, at least, two types of computer-readable media, namely computer storage media and communication media. Computer storage media includes volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD), Blu-Ray, or other optical storage, magnetic cassettes, magnetic tape, magnetic disk storage or other magnetic storage devices, or any other non-transmission medium that may be used to store information for access by a computing device. In contrast, communication media may embody computer-readable instructions, data structures, program modules, or other data in a modulated data signal, such as a carrier wave, or other transmission mechanisms. As defined herein, computer storage media does not include communication media.

The service provider platform 104 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. The service provider platform 104 can include one or more virtual machines running virtual operating systems, or other computing architectures involving virtualization such as one or more flexible pools of logical storage devices that can be virtualized to maintain virtual storage devices for the computer.

The service provider may maintain one or more software modules configured to perform at least a portion of the functionality described herein. In accordance with at least some embodiments, the service provider platform 104 may provide processing of a material sample as collected by the sample collection device 102 via a sample analysis engine 114. This may comprise providing images of the sample obtained from the sample collection device 102 to a trained machine learning model 116 as input. In this process, the trained machine learning model 116 may be any suitable machine learning algorithm that has been trained to correlate image data for a particular type of material sample with one or more conditions attributable to the material sample. In some embodiments, separately trained machine learning models may be maintained for each different type of material sample that may be analyzed. Each trained machine learning model may have access to a data store (e.g., condition data 118) that includes information on one or more conditions that might be associated with a particular type of material sample (e.g., disease symptoms, identified abnormalities, etc.).

Upon receiving one or more images associated with a material sample, the sample analysis engine 114 may be configured to perform object recognition to identify one or more objects depicted within the images. The objects may be identified within the images based on the detected characteristics of those objects. For example, blood cells may be identified based on their shape, size, and/or color as compared to information known about blood cells. In this example, upon identifying blood cells within the material sample, a determination may be made that the material sample is a blood sample. The information known about blood cells may include information about multiple different types of red blood cells and/or multiple different types of white blood cells. For example, information known about red blood cells may include, by way of non-limiting example, normal cells, sickle cell, bite cells, burr cells, and/or basophilic cells. In another example, information known about white blood cells may include, by way of non-limiting example, neutrophils, eosinophils, basophils, monocytes, and B and T lymphocytes.

Subsequently, the sample analysis engine 114 may identify a condition to be associated with the material sample. In some cases, one or more conditions may be determined based on identified characteristics of the material sample. For example, upon determining that a material sample comprises a blood sample, the sample analysis engine 114 may identify and count red blood cells, white blood cells, and/or platelets. In some cases, a diagnosis may be made based on such a count. In other cases, a diagnosis may be made based on variances in a detected shape of the object in the material or via a comparison to an object affected by that condition.

Such a condition may include an indication that the sample is normal or an indication that the sample is abnormal as well as a likely type/category of abnormality to be attributed to the sample. The sample analysis engine 114 may be configured to report out the determination of the trained machine learning. In some embodiments, such a determination may take the form of a diagnosis and a respective likelihood or confidence value. The report out may be made to an account or contact associated with an entity from which the material sample is determined to have been received. For example, the entity may be a user from which the material sample is collected. In some embodiments, upon identifying a type or category of a condition associated with the material sample, the images associated with that material sample may be provided to another party, such as an expert or administrator, for a second opinion. Information provided by the party in response to receiving the images may then be used to further refine the trained machine learning model 116.

In some embodiments, the sample analysis engine 114 may be further configured to provide feedback to be used to refine the trained machine learning model 116 included on the sample collection device 102. For example, upon receiving images of a material sample, a determination may be made that the material sample is not the type of sample that it was determined to be. In this example, correction information (e.g., the images and an indication of a correct sample type) may be added to a data store of input/output data 120 that includes data to be used to train the machine learning model 110.

In some embodiments, the service provider platform 104 may maintain a software module configured to train/generate a machine learning model to be included in the memory of one or more sample collection devices (e.g., a model generation engine 122). In some cases, a model generation engine may periodically generate or update a machine learning model to determine a type of material sample based on visual characteristics of that sample. To do this, the model generation engine may provide a set of images associated with various material samples to a machine learning algorithm as input as well as an indication of categories or types of the material sample as output. The machine learning algorithm may then be “trained” to correlate the inputs to the outputs. In some cases, this may comprise adjusting variables or weights within an algorithm until the inputs result in the appropriate outputs. The model generation engine may be further configured to provision one or more trained machine learning models onto a sample collection device. In some cases, the model generation engine may update or re-generate a trained machine learning model as new input/output data is received or on a periodic basis.

Various interactions may occur between the described components of the system 100. In the system 100, a user may interact with the sample collection device 102 to provide a material sample for analysis. In this example, the user may place the material sample into a collection cartridge 108 configured to receive such a sample. The collection cartridge 108 may then be placed into the sample collection device 102 for processing.

Upon receiving a collection cartridge 108 that includes a material sample, the sample collection device 102 may be configured to obtain at least one initial image of the material sample. Upon obtaining such an image, the sample collection device 102 may compare the visual characteristics of the material sample with those of potential sample categories/types in order to determine a category or type to which the sample likely belongs. Once a likely sample type has been determined (e.g., using the trained machine learning model 110), a set of data collection instructions may be identified as being associated with the determined sample type. Those data collection instructions may then be used to collect a set of images that may be used to analyze the material sample. For example, the data collection instructions may indicate a number of images to be collected as well as a magnification, lighting (e.g., level, color, wavelength, etc.) to be used when obtaining the images, which testing compartments to collect images from (when the collection cartridge 108 includes multiple testing compartments), etc. The sample collection device 102 then collects the appropriate images in accordance with the data collection instructions and provides the collected images to the service provider platform 104.

Upon receiving the images collected by the sample collection device 102, the service provider platform 104 may provide those images to another trained machine learning model as input. In one example, the service provider platform 104 may provide the received images to a machine learning model (e.g., trained machine learning model 116) that has been trained to determine one or more conditions associated with the particular type of material sample that is being analyzed. In a second example, the service provider platform 104 may provide the received images to a machine learning model that has been trained to identify a particular condition within the material sample. For example, the images may be provided to a machine learning model that has been trained to differentiate between normal blood cells and sickle-cell blood cells. Once the trained machine learning model has been provided with the images as input, the output generated by such a model may be used to provide a diagnosis.

The illustrative system includes a service provider platform 104. However, it should be understood that a service provider platform 104 can be several computers, layers, or other elements, processes or components, which may be chained or otherwise configured, which can interact to perform tasks such as obtaining data from an appropriate data store. As used herein the term “data store” refers to any device or combination of devices capable of storing, accessing, and retrieving data, which may include any combination and number of data servers, databases, data storage devices and data storage media, in any standard, distributed or clustered environment. The service provider platform 104 can include any appropriate hardware and software for integrating with the data store as needed to execute aspects of one or more applications for the sample collection device 102, handling a majority of the data access and business logic for an application installed upon (and executed from) the sample collection device 102. The service provider platform 104 provides access control services in cooperation with the data store and is able to generate content such as text, graphics, audio and/or video to be transferred to the sample collection device 102, which may be served to the user in the form of HyperText Markup Language (“HTML”), Extensible Markup Language (“XML”) or another appropriate structured language in this example.

For clarity, a certain number of components are shown in FIG. 1. It is understood, however, that embodiments of the disclosure may include more than one of each component. In addition, some embodiments of the disclosure may include fewer than or greater than all of the components shown in FIG. 1. In addition, the components in FIG. 1 may communicate via any suitable communication medium, using any suitable communication protocol. In some cases, one or more of the components may operate on a network. A network can include any appropriate network, including an intranet, the Internet, a cellular network, a local area network or any other such network or combination thereof. Components used for such a system can depend at least in part upon the type of network and/or environment selected. Protocols and components for communicating via such a network may be known to one skilled in the art and will not be discussed herein in detail. Communication over the network can be enabled by wired or wireless connections and combinations thereof.

In some instances, data regarding conditions that that are diagnosed based on the material samples collected from various users may be collated and anonymized by a data analytics engine 124 of the service provider platform 104 for further anonymized data analysis. For example, the data analytics engine 124 of the service provider platform 104 may provide anonymized data analytics services to various third parties, such as government agencies, industry groups, community health organizations, and/or so forth. The anonymized data analytics services may identify disease or sickness trends that affect different communities, population segments, etc. based on the anonymized data regarding identified conditions. For example, when a disease or sickness trend may be detected by the data analytics engine 124 for a particular community, such information may be provided to a local government agency or healthcare organization so that the agency or organization responds to a potential medical or community concern. In instances in which the data analytics engine 124 receives additional demographic and/or lifestyle information regarding the various users, the data analytics engine 124 may further analyze and present anonymized information regarding the disease or sickness trends according to various demographic and/or lifestyle variables, such as age, sex, sexuality, race, diet, drug use, and/or so forth in a community.

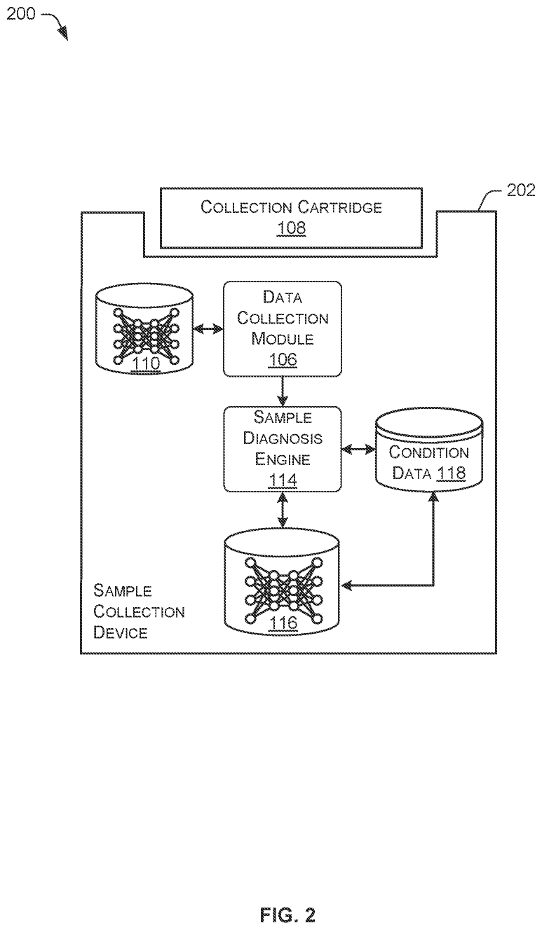

FIG. 2 depicts an example of a second illustrative system 200 that may be implemented in accordance with various embodiments of the disclosure. In the system depicted in FIG. 2, a diagnosis for a material sample may be generated locally by a sample collection device 202.

Similar to the sample collection device 102 of FIG. 1, a sample collection device 202 may be configured to receive a collection cartridge 108. As described elsewhere, the sample collection device 202 may include at least a data collection module 106 that facilitates collection of material samples by the sample collection device 202 as well as at least one machine learning model 110 that has been trained to correlate detected sample information with a number of data collection information/requirements.

Unlike the sample collection device 102, the sample collection device 202 may further include a sample analysis engine 114, a trained machine learning model 116, and condition data 118. Each of these components may be equivalents of the respective sample analysis engine 114, trained machine learning model 116, and condition data 118 of FIG. 1. It should be noted that the trained machine learning model 116 may be generated on a server or other remote computing device and provisioned onto a local memory of the sample collection device 202.

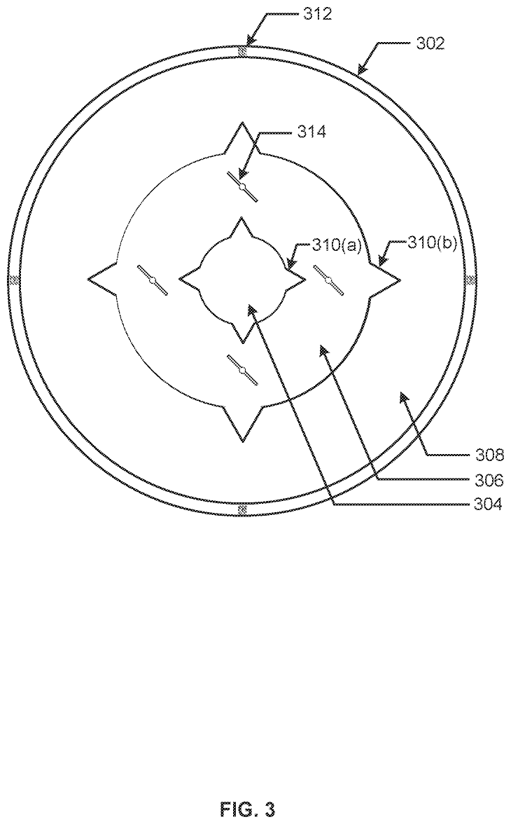

FIG. 3 depicts a first example of a collection cartridge having a single testing compartment that may be used along with a sample collection device in accordance with at least some embodiments. As depicted in FIG. 3, a collection cartridge 302 may include multiple areas that facilitate testing and analysis of a material sample. By way of example, the collection cartridge 302 may include a sample collection area 304, a mixing area 306, and an imaging and measurement area 308. Each of the areas may be separated by one or more valves 310 (e.g., valve 310(a) and valve 310(b)).

The collection cartridge 302 may be configured to be received upon a platter of the sample collection device 102 that is capable of spinning in order to apply centrifugal force to the received collection cartridge 302. In some embodiments, the width of one or more areas of the collection cartridge 302 may be configured to receive the material sample in a manner that is most optimal for analysis. For example, the width/depth of the imaging area might be 0.1 ml in order to limit the amount of fluid located in the imaging area and to optimize imaging of that fluid.

In some embodiments, the collection cartridge 302 may include one or more indicators 312 that may be used to identify the collection cartridge 302, a user associated with the collection cartridge 302 and/or a position within the collection cartridge 302. In some embodiments, such an indicator may take the form of a machine-readable code that can be interpreted using one or more machine vision techniques. In some embodiments, such a machine-readable code may comprise a barcode or a quick response (QR) code.

In the depicted example collection cartridge 302, a material sample is inserted into the sample collection area 304. When centrifugal force is applied to the collection cartridge 302 by spinning the cartridge, the inserted material sample is pushed to the exterior of the sample collection area 304 and through the valve 310(a) into the mixing area 306. In some embodiments, the mixing area 306 may include a diluent or other reagent to be mixed with the material sample in order to facilitate analysis. In some embodiments, the mixing area 306 may further include a number of aggregators 314 that are configured to facilitate mixing of the diluent with the material sample. The aggregators 314 may be protrusions from the bottom of the collection cartridge 302 that extend upright into the mixing area 306. In some embodiments, the aggregators 314 are fixed in their respective positions with respect to the sample collection cartridge. Thus, as the mixing chamber of the collection cartridge 302 is caused to rotate, the aggregators 314 facilitate mixing of the material sample and the diluent in the mixing area 306 as the material sample and the diluent moves around the aggregators 314.

When the material sample is pushed into the mixing area 306, it is mixed with the diluent included within that mixing area 306. The centrifugal force continues to push the mixture including the material sample and the diluent to the outside of the mixing area 306, through a second valve 310 (b), and into the imaging area. When the mixture has entered the imaging area, one or more images of the mixture may be obtained by the sample collection device.

In this example collection cartridge 302, measurement would be conducted by imaging of a fixed volume of the imaging area contents in order to extrapolate the total volume of the sample. In some cases, a fixed amount of diluent is included within the mixing chamber based on a predicted likely volume of the material sample. As an example, red blood cell (RBC) count is typically conducted with a ratio of 1 part blood to 200 parts diluent. A single drop of blood is estimated at 50 uL, however, can vary greatly. Accordingly, a recommended amount of diluent to be included in the mixing area 306 might be 10,000 uL or 10 ml for a predicted 50 ul blood sample. In some embodiments, the sample collection area 304 may allow for a specified amount of sample (e.g., 5 uL of blood), to be moved to the mixing chamber and combined with a specific amount (e.g., 995 ul) of diluent to achieve a predetermined ratio (e.g., 1:200 sample to diluent). In these embodiments, a much smaller total volume could be packed into a total cartridge diameter of 45 mm (˜1.75 in).

In some cases, the diluent ratio may be reduced to accommodate a smaller collection cartridge in turn affecting the calculation for RBC count. In this example, a blood sample of 60 ul would be slightly larger than normal and would change the ratio from 1:200 (50:10,000) to 1:166.6 (60:10,000). Accordingly, this would require the count of RBC to be adjusted to consider a more concentrated number of RBCs.

One or more portions of the collection cartridge 302 may be constructed of transparent material in order to enable images to be captured of the materials (including the material sample and any reagents) located within the cartridge. In some embodiments, the one or more transparent portions may be shaped in a manner that facilitates magnification of the materials within the testing compartment. For example, a transparent portion may comprise a lens that includes a convex or concave surface.

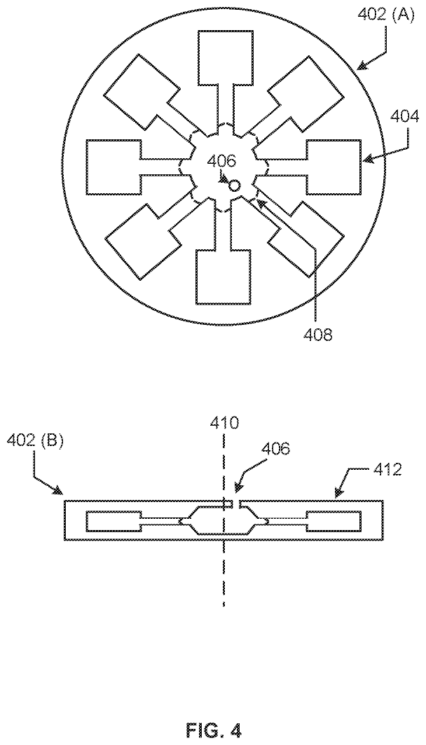

FIG. 4 depicts a second example of a collection cartridge having multiple testing compartments that may be used along with a sample collection device in accordance with at least some embodiments. Depicted in FIG. 4 is an exemplary collection cartridge 402, where 402(A) represents a top-down view of the collection cartridge and 402(B) represents a side view of the collection cartridge 402. It should be noted that the collection cartridge 402 depicted in FIG. 4 is exemplary only and other collection cartridges that accomplish the same results might also be within the scope of the disclosure.

As depicted at 402(A) the collection cartridge 402 may be circular in shape in order to facilitate rotational movement around an axis 410. For example, a sample collection device into which the collection cartridge 402 is to be placed may be configured to spin the collection cartridge 402 in order to introduce centrifugal force or to position the collection cartridge 402 for optimal image collection. In some embodiments, the collection cartridge 402 may include a number of testing compartments (or chambers) 404. These testing compartments 404 may be located along the exterior of the circular collection device.

The collection cartridge 402 may include a sample insertion port 406 into which a material sample may be placed for analysis. In some embodiments, the sample insertion port 406 may be located near a center portion of the collection cartridge 402, but offset from the center in order to facilitate movement of the material sample via centrifugal force. A material sample may be added to the collection cartridge 402 via a needle or other sample collection device inserted into the sample insertion port 406.

Each of the testing compartments 404 may be accessible via a valve 408 that allows flow of the material sample into the respective testing compartment. In some embodiments, the valve 408 is a one-way valve that allows liquids to flow in a single direction (e.g., into the testing compartment). One or more of the testing compartments 404 may include a reagent or other substance that facilitates or otherwise enables the performance of a particular test. When the collection cartridge 402 is spun with enough force, the material sample may be pushed to the exterior of the collection cartridge 402, and subsequently may be pushed through one or more valves and into a respective testing compartment.

One or more portions 412 of the collection cartridge 402 may be constructed of transparent material in order to enable images to be captured of the materials (including the material sample and any reagents) located in a testing compartment. In some embodiments, the transparent portion 412 may be shaped in a manner that facilitates magnification of the materials within the testing compartment. For example, the transparent portion 412 may comprise a lens that includes a convex or concave surface.

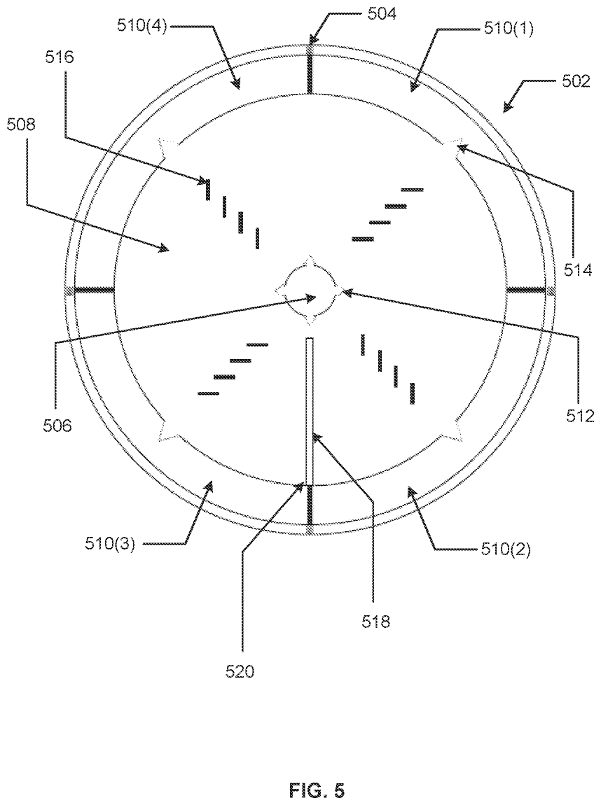

FIG. 5 depicts a third example of a collection cartridge that may be used along with a sample collection device in accordance with at least some embodiments. As depicted in FIG. 5, a collection cartridge 502 may include multiple areas that facilitate testing and analysis of a material sample. In some embodiments, the collection cartridge 502 may include one or more indicators 504 that may be used to identify the collection cartridge 502, a user associated with the collection cartridge 502 and/or a position within the collection cartridge 502. In some embodiments, such an indicator may take the form of a machine-readable code that can be interpreted using one or more machine vision techniques. In some embodiments, such a machine-readable code may comprise a barcode or a quick response (QR) code.

The collection cartridge 502 may be configured to be received upon a platter of the sample collection device 102 that is capable of spinning in order to apply centrifugal force to the received collection cartridge 502. By way of example, the collection cartridge 502 may include a sample receptacle area 506, a mixing area 508, and dye areas 510(1)-510(4). Each of the areas may be separated by one or more one-way valves. For example, the sample receptacle area 506 may be separated from the mixing area 508 by one or more one-way valves (e.g., valve 512) that only permit the flow of material from the sample receptacle area 506 into the mixing area 508. Likewise, the mixing area 508 may be separated from each of the dye areas 510(1)-510(4) by a corresponding one-way valve that only permits the flow of material from the mixing area 508 to a corresponding dye area. For example, the dye area 510(1) may be separated from the mixing area 508 by a one-way valve 514.

In some embodiments, a material sample (e.g., a 5 uL blood sample) is inserted into the sample receptacle area 506. When centrifugal force is applied to the collection cartridge 502 by spinning the cartridge, the inserted material sample is pushed to the exterior of the sample receptacle area 506 and through the valves (e.g., valve 512) into the mixing area 508. In some embodiments, the mixing area 508 may include a diluent or other reagent (e.g., normal saline solution) to be mixed with the material sample in order to facilitate analysis. The spin speed and the spin time of the collection cartridge 502 may be controlled such that the amount of material sample and the amount of diluent or reagent achieves a predetermined ratio. In some embodiments, the mixing area 508 may further include a number of aggregators, such as the aggregators 516, that are configured to facilitate the mixing of the diluent with the material sample. The aggregators may be protrusions from the bottom of the collection cartridge 502 that extend upright into the mixing area 508. In some embodiments, the aggregators are fixed in their respective positions with respect to the sample collection cartridge. Thus, as the mixing chamber of the collection cartridge 502 is caused to rotate, the aggregators facilitate the mixing of the material sample and the diluent in the mixing area 508 as the material sample and the diluent moves around the aggregators. Accordingly, when the material sample is pushed into the mixing area 508, it is mixed with the diluent included within that mixing area 508.

Subsequently, one or more images of the resultant mixture may be obtained by the sample collection device for further measurements and/or analysis. For example, the various measurements and/or analyses that may be performed on a resultant mixture that contains blood may include an RBC count, a WBC count, a platelet count, RBC morphology, mean corpuscular volume (MCV), red cell distribution width (RDW), and/or so forth. Further, with the application of a corresponding color filter over the image capture device that filters out light of specific wavelengths, additional measurements and/or analyses that may be performed on the resultant mixture via imaging may include a mean corpuscular hemoglobin (MCH) test, a mean corpuscular hemoglobin concentration (MCHC) test, other tests related to hemoglobin, and/or so forth.

Once the imaging is performed with respect to the mixture in the mixing area 508, the collection cartridge 502 may be further spun at a higher rotational speed to continue to push the mixture including the material sample and the diluent to the outside of the mixing area 508, and through corresponding one-way valves (e.g., the valve 514), into the dye areas 510(1)-510(4). The one-way valves leading into the dye area 510(1)-510(4), such as the valve 514, may be configured to require a higher force to open than the one-way valves (e.g., the valve 512) between the sample receptacle area 506 and the mixing area 508. When the mixture has entered a dye area, at least some of the material sample in the mixture may be stained by a dye that is present in the dye area. Subsequently, one or more images of the mixture may be obtained by the sample collection device of the dye area for further measurements and/or analysis. For example, a dye in a dye area may stain certain cells in a blood sample such that a white blood cell (WBC) differential test may be performed based on the one or more images. In this way, the dyes in the dye areas 510(1)-510(4) may enable the material sample to be dyed with different colored dyes, such that the images of the corresponding stained mixtures may be captured by the sample collection device for subsequent measurements and/or analysis.

The collection cartridge 502 may be further equipped with a tube receptacle for holding a sample tube 518. The tube receptacle may be a receiving groove on the bottom of the collection cartridge 502 in which the sample tube 518 is removably fitted and frictionally held in place by the sides of the groove. The tube receptacle may be positioned along a radius of the collection cartridge 502. The sample tube 518 may be a tube that is made from a transparent material. The tube openings at the ends of the sample tube 518 may be sealed after a material sample (e.g., a 5 uL blood sample) is deposited inside the tube via one or more of the tube openings. Following the deposit of the material sample into the sample tube 518, the sample tube 518 may be inserted into the tube receptacle of the collection cartridge 502 prior to the spinning of the collection cartridge 502. As the collection cartridge 502 is spun at a predetermined rotational speed for a predetermined period of time, the centrifugal force provided by the spinning of the collection cartridge 502 may move the heavier materials in the material sample (e.g., heavier cells in a blood sample) towards the distal end 520 of the sample tube 518. Once the heavier materials have moved to the distal end 520, one or more images of the sample tube 518 may be captured by the sample collection device for further measurements and/or analyses. For example, the measurements and/or analyses may include a hematocrit test on the blood in the sample tube 518.

One or more portions of the collection cartridge 502 may be constructed of transparent material in order to enable images to be captured of the materials (including the material sample and any reagents) located within the cartridge. In some embodiments, the one or more transparent portions may be shaped in a manner that facilitates magnification of the materials within the testing compartment. For example, a transparent portion may comprise a lens that includes a convex or concave surface.

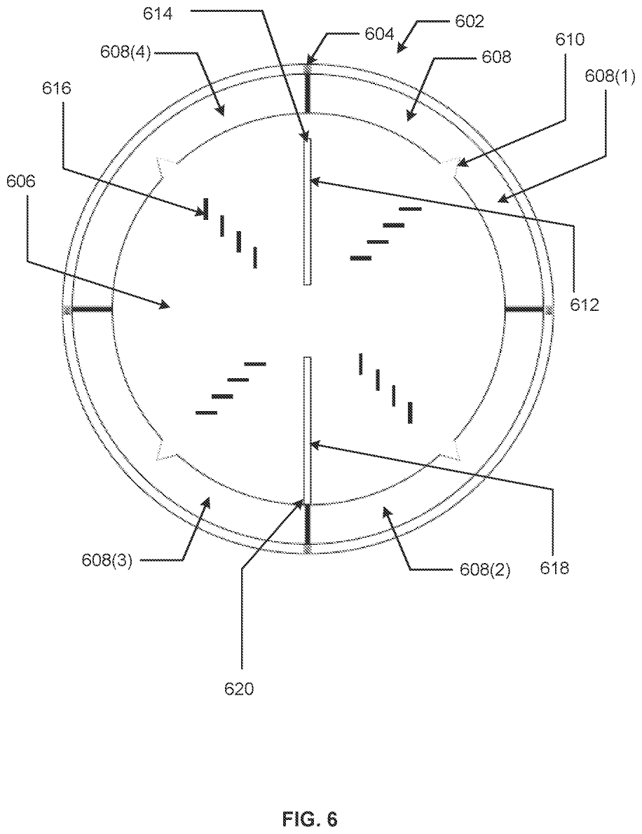

FIG. 6 depicts a fourth example of a collection cartridge that may be used along with a sample collection device in accordance with at least some embodiments. As depicted in FIG. 6, a collection cartridge 602 may include multiple areas that facilitate testing and analysis of a material sample. In some embodiments, the collection cartridge 602 may include one or more indicators 604 that may be used to identify the collection cartridge 602, a user associated with the collection cartridge 602 and/or a position within the collection cartridge 602. In some embodiments, such an indicator may take the form of a machine-readable code that can be interpreted using one or more machine vision techniques. In some embodiments, such a machine-readable code may comprise a barcode or a quick response (QR) code.

The collection cartridge 602 may be configured to be received upon a platter of the sample collection device 102 that is capable of spinning in order to apply centrifugal force to the received collection cartridge 502. By way of example, the collection cartridge 602 may include a mixing area 606, and dye areas 608(1)-608(4). The mixing area 606 may be separated from each of the dye areas 608(1)-608(4) by a corresponding one-way valve that only permits the flow of material from the mixing area 606 to a corresponding dye area. For example, the dye area 608(1) may be separated from the mixing area 606 by a one-way valve 610.

The collection cartridge 602 may be further equipped with a tube receptacle for holding a sample tube 612. The tube receptacle may be a receiving groove on the bottom of the collection cartridge 602 in which the sample tube 612 is removably fitted and frictionally held in place by the sides of the groove. The tube receptacle may be positioned along a radius of the collection cartridge 602. The sample tube 612 may be a tube that is made from a transparent material. A material sample (e.g., a 5 uL blood sample) may be deposited inside the tube via one or more of the open ends of the sample tube 612. Following the deposit of the material sample into the sample tube 612, the sample tube 612 may be inserted into the tube receptacle of the collection cartridge 602 prior to the spinning of the collection cartridge 602. As the collection cartridge 602 is spun at a predetermined rotational speed for a predetermined period of time, the centrifugal force provided by the spinning of the collection cartridge 602 may push the material sample out of a tube opening at the distal end 614 of the sample tube 612 into the mixing area 606.

The mixing area 606 may include a diluent or other reagent (e.g., normal saline solution) to be mixed with the material sample in order to facilitate analysis. The spin speed and the spin time of the collection cartridge 602 may be controlled such that the amount of material sample and the amount of diluent or reagent achieves a predetermined ratio. In some embodiments, the mixing area 606 may further include a number of aggregators, such as the aggregators 616, that are configured to facilitate the mixing of the diluent with the material sample. The aggregators may be protrusions from the bottom of the collection cartridge 602 that extend upright into the mixing area 606. In some embodiments, the aggregators are fixed in their respective positions with respect to the sample collection cartridge. Thus, as the mixing chamber of the collection cartridge 602 is caused to rotate, the aggregators facilitate the mixing of the material sample and the diluent in the mixing area 606 as the material sample and the diluent moves around the aggregators. Accordingly, when the material sample is pushed into the mixing area 606, it is mixed with the diluent included within that mixing area 606.

Subsequently, one or more images of the resultant mixture may be obtained by the sample collection device for further measurements and/or analyses. For example, the various measurements and/or analyses that may be performed on a resultant mixture that contains blood may include an RBC count, a WBC count, a platelet count, RBC morphology, MCV, RDW, and/or so forth. Further, with the application of a corresponding color filter over the image capture device that filters out light of specific wavelengths, additional measurements and/or analyses that may be performed on the resultant mixture via imaging may include a MCH test, a MCHC test, other tests related to hemoglobin, and/or so forth.

Once the imaging is performed with respect to the mixture in the mixing area 606, the collection cartridge 602 may be further spun at a higher rotational speed to continue to push the mixture including the material sample and the diluent to the outside of the mixing area 606, and through corresponding one-way valves (e.g., the valve 610), into the dye areas 608(1)-608(4). When the mixture has entered a dye area, at least some of the material sample in the mixture may be stained by a dye that is present in the dye area. Subsequently, one or more images of the mixture may be obtained by the sample collection device of the dye area for further measurements and/or analyses. For example, a dye in a dye area may stain certain cells in a blood sample such that a WBC differential test may be performed based on the one or more images. In this way, the dyes in the dye areas 608(1)-608(4) may enable the material sample to be dyed with different colored dyes, such that the images of the corresponding stained mixtures may be captured by the sample collection device for subsequent measurements and/or analyses.

The collection cartridge 602 may be further equipped with an additional tube receptacle for holding a sample tube 618. The tube receptacle may be a receiving groove on the bottom of the collection cartridge 602 in which the sample tube 618 is removably fitted and frictionally held in place by the sides of the groove. The tube receptacle may be positioned along a radius of the collection cartridge 602. The sample tube 618 may be a tube that is made from a transparent material. The openings at the ends of the sample tube 618 may be sealed after a material sample (e.g., a 5 uL blood sample) is deposited inside the tube via one or more of the openings. Following the deposit of the material sample into the sample tube 618, the sample tube 618 may be inserted into the tube receptacle of the collection cartridge 602 prior to the spinning of the collection cartridge 602. As the collection cartridge 602 is spun at a predetermined rotational speed for a predetermined period of time, the centrifugal force provided by the spinning of the collection cartridge 602 may move the heavier materials in the material sample (e.g., heavier cells in a blood sample) towards the distal end 620 of the sample tube 618. Once the heavier materials have moved to the distal end 620, one or more images of the sample tube 618 may be captured by the sample collection device for further measurements and/or analyses. For example, the measurements and/or analyses may include a hematocrit test on the blood in the sample tube 618.

One or more portions of the collection cartridge 602 may be constructed of transparent material in order to enable images to be captured of the materials (including the material sample and any reagents) located within the cartridge. In some embodiments, the one or more transparent portions may be shaped in a manner that facilitates magnification of the materials within the testing compartment. For example, a transparent portion may comprise a lens that includes a convex or concave surface.

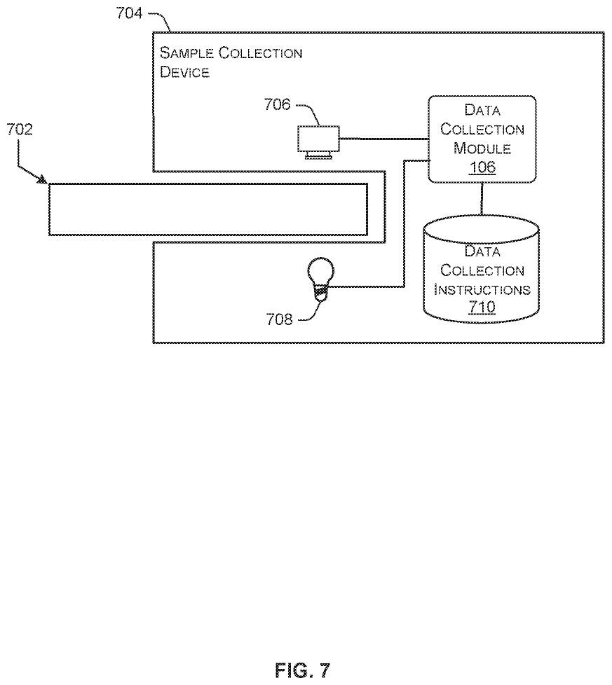

FIG. 7 depicts a block diagram illustrating various features of a sample collection device in accordance with at least some embodiments. As depicted in FIG. 7, a collection cartridge 702 may be inserted into a receiving slot of a sample collection device 704. The sample collection device 704 may be an embodiment of the sample collection device 102 illustrated in FIG. 1 or an embodiment of the sample collection device 202 illustrated in FIG. 2. In some embodiments, when the collection cartridge 702 is properly positioned, the sample collection device may be configured to spin the collection cartridge 702 in order to apply a centrifugal force to the collection cartridge 702 (e.g., to move the material sample into the testing compartments) or to select a specified testing compartment for analysis.

The sample collection device 704 may include an image capture device 706 (e.g., a camera) that is configured (when the collection cartridge 702 is properly positioned) to capture images of a material sample that is located within a testing compartment of the collection cartridge 702. In some embodiments, the sample collection device 704 may include a light source 708 that may be positioned to provide lighting from a side of the collection cartridge 702 that is opposite the one on which the image capture device is located. The sample collection device 706 may be equipped with one or more lenses that focus and collect light from the light source 708. For example, the one or more lenses may include a condenser lens that focuses light from the light source 708 onto the material sample, and the image capture device 706 may be equipped with an objective lens that collects light from the material sample and provides a magnified image that includes at least a portion of the material sample to the image capture device 706. Accordingly, the image capture device 706 and the light source 708 may be used in combination to perform bright field microscopy on the material sample that is within a test compartment of the collection cartridge 702. In other embodiments, the sample collection device 704 may be configured to capture dark field microscopy images of the material sample. For example, the light source 708 may be provided with a condenser lens that is configured with a direct illumination attenuator (e.g., an opaque disc) that blocks directly transmitted light of the light source 708 from entering the objective lens, while permitting light scattered by the material sample to enter an objective lens that provides the scattered light to the image capture device 706 for performing dark field microscopy.

In additional embodiments, the sample collection device 704 may be further configured to capture fluorescence microscopy images of the material sample. For example, the light source 708 may be configured to provide light with one or more specific wavelengths that cause emission of fluorescence from one or more portions of the material sample after the absorption of the light of the one or more specific wavelengths by the material sample. The fluorescence from the one or more portions of the material sample may be captured by the objective lens of the image capture device 706 instead of or in addition to the scattered or reflected lights of other wavelengths such that the image capture device 706 may capture images that include the one or more fluorescent portions of the material sample. The image capture device 706, the light source 708, and/or other features of the sample collection device 704, may be controlled by the data collection module 106 executed on the sample collection device 704.

In some embodiments, the data collection module 106 may be configured to identify a set of data collection instructions in accordance with a data collection strategy identified for the particular material sample being analyzed. In some embodiments, the sample collection device 704 may store such data collection instructions within a data store (e.g., data collection instructions 710) that houses data collection instructions for each of a number of material sample types. In some embodiments, an assessment may first be made as to what type of material sample is being analyzed. In some embodiments, this comprises collecting an initial image of the material sample (which may be obtained from a specified testing compartment of the collection cartridge 702) and providing that initial image to a trained machine learning model. In some embodiments, this comprises receiving an indication of a type or category of the material sample from a user of the sample collection device 704 or a machine-readable code associated with the material sample. Upon assessing the type or category for the material sample, data collection instructions for that type or category may be retrieved from the data store of data collection instructions.

Data collection instructions retrieved in relation to the material sample may include any suitable instructions for obtaining information about the material sample. For example, the data collection instructions may include an indication of how many images are to be collected, which testing compartments are to be imaged, what magnification level/settings are to be used during such imaging, what type/intensity of light is to be used (e.g., via light source 708), the type of microscopy technique to be used (e.g., bright field, dark field, fluorescence, etc.), the number of images to be captured using each type of microscopy technique, and/or any other suitable instructions. In some instances, bright field microscopy may be used for imaging a majority of material samples, while other material samples may be imaged using dark field microscopy or fluoresce microscopy. In other instances, some material samples may be imaged using a combination of multiple microscopy techniques. For example, a material sample may be imaged via bright field microscopy for a first predetermined number of times, imaged via dark field microscopy for a second predetermined number of times, and/or imaged via fluorescence microscopy for a third predetermined number of times. With respect to the imaging via fluoresce microscopy, the data collection instructions may specify different wavelengths of light for different images to be captured such that different types of fluorophores in a material sample that fluorescence due to different wavelengths of light may be captured in the different images of the material sample.

Once data collection instructions have been retrieved in relation to a particular material sample, those data collection instructions may be executed. For example, the data collection instructions may be executed to perform a complete blood count, an RBC count, a white blood cell (WBC) count, a hemoglobin count, hematocrit, a WBC differential test, and/or so forth. In some cases, this may comprise spinning the collection cartridge 702 until a specified testing compartment is aligned with an image capture device 706 of the sample collection device 704. Upon aligning the testing compartment, the sample collection device 704 may activate the light source 708 and capture an image of the material sample within the aligned testing compartment. This may be repeated a number of times (e.g., once for each of the received data collection instructions) and the resulting captured images may then be transmitted by the sample collection device to a service provider platform 104 via a wired or wireless communication channel.

In some alternative embodiments, the collection cartridges holding the material samples may be substituted with microscope slides. In such embodiments, the image capture device 706 (e.g., a camera) of the sample collection device 704 may be configured to capture images of material samples that are located within standard microscope slides. Each of the standard microscope slides may have an overall width of approximately 76.2 mm (3 inches) and an overall length of approximately 25.4 mm (1 inch). The microscope slide may have a material imaging area for holding the material sample for imaging by the image capture device 706. The material image area may be an area with a length and width of approximately 22 mm, and the area is centered lengthwise and widthwise on the microscope slide. The microscope slides may be positioned into a receiving slot of a microscope stage underneath the image capture device. Accordingly, the image capture device 706 may be configured to image the material imaging areas of the microscope slides by using one or more motorized actuators to automatically move over the material image areas of the microscope slides and capture images of the material samples.

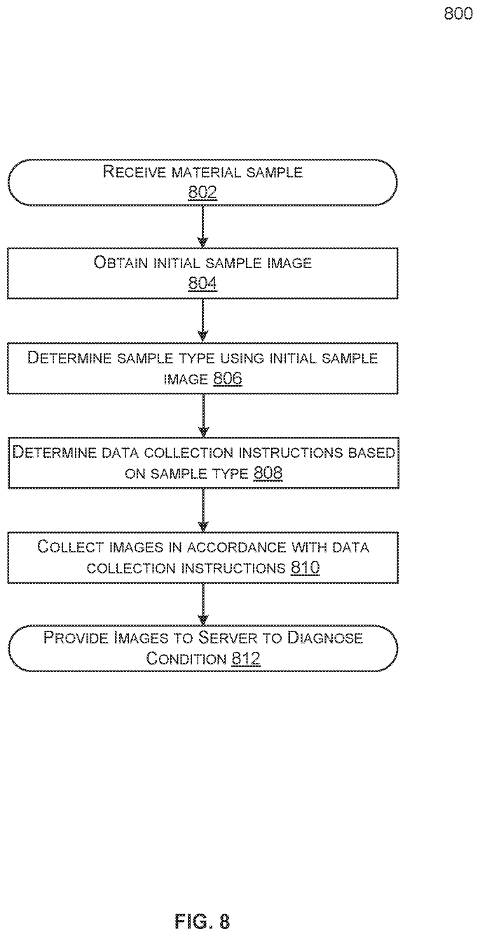

FIG. 8 depicts a flow diagram illustrating an exemplary process 800 for determining a sample collection strategy and collecting images in accordance with embodiments. The process 800 is illustrated as a logical flow diagram, each operation of which represents a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be omitted or combined in any order and/or in parallel to implement this process and any other processes described herein.

Some or all of the exemplary process 800 (or any other processes described herein, or variations and/or combinations thereof) may be performed under the control of one or more computer systems configured with executable instructions and may be implemented as code (e.g., executable instructions, one or more computer programs or one or more applications). In accordance with at least one embodiment, the process 800 of FIG. 8 may be performed by one or more computing devices as shown in FIG. 1. For example, the process 800 may be performed by a sample collection device 102 or by a service provider platform 104 as described with respect to FIG. 1. The code may be stored on a computer-readable storage medium, for example, in the form of a computer program including a plurality of instructions executable by one or more processors. The computer-readable storage medium may be non-transitory.

At block 802, process 800 begins upon receiving a material sample to be analyzed. In some embodiments, the material sample may be included within a collection cartridge that is received within a slot or other receiver means of the sample collection device. In some embodiments, such a collection cartridge is configured to be rotated by the sample collection device around an axis. It should be noted that while rotation is described herein, rocking or vibrating the collection cartridge may serve an equivalent function. In at least a portion of these embodiments, the sample collection device may rotate the collection cartridge to introduce centrifugal force in order to move the sample toward the exterior of the collection cartridge (and potentially into one or more testing compartments). Alternatively, the collection cartridge may be rotated in order to position a selected testing compartment of the collection cartridge so that its contents can be imaged.

At block 804, the process 800 comprises obtaining at least one initial image of the material sample. In some embodiments, the sample collection device may be configured to obtain the initial image from a predetermined testing compartment of the collection cartridge and using predetermined settings (e.g., level of magnification, lighting, etc.).

At block 806, the process 800 comprises determining a category for the sample. A sample category may include any suitable delineation by which a sample may be categorized. For example, a material sample may be categorized as a “blood sample,” a “semen sample,” a “saliva sample,” etc. In some embodiments, a category is determined for the sample by providing the initial image of the sample to a machine learning model that has been trained to correlate image data with sample types.

At block 808, the process 800 comprises determining a set of data collection instructions based on the sample type. In some embodiments, the sample collection device stores a number of sets of data collection instructions. In some cases, each of those sets of data collection instructions relates to a different sample type. Each set of data collection instructions may include an indication of how many and which images to capture in relation to a sample. Such instructions may indicate a level of magnification, a light setting, a testing compartment identifier, or any other suitable indication as to how an image should be captured.

At block 810, the process 800 comprises collecting images in accordance with the retrieved data collection set. The sample collection device may obtain at least one image for each data collection instruction in the retrieved set of data collection instructions. To do this, the sample collection device may cause a collection cartridge to be rotated until a specified testing compartment is positioned under an image capture device. The sample collection device may then adjust a level of magnification (e.g., zoom), light level/type, microscopy technique, and/or so forth to match that indicated in the data collection instructions. Once each of the specified settings indicated in the data collection instructions has been met, an image is captured via the image capture device. This is repeated for each of the data collection instructions in the set of data collection instructions in order to generate a set of images.

At block 812, the process 800 comprises providing the set of collected images to be analyzed (e.g., locally or via service provider platform 104) so that a diagnosis can be made. In some embodiments, the sample collection device may provide, along with the set of collected images, an indication of the determined sample type. In some cases, the diagnosis is made by the server by providing the set of images to a second machine learning model that has been trained to correlate image data with particular conditions. In some embodiments, separate machine learning models may be maintained for each of multiple sample types.

A diagnosis made with respect to the sample may be provided to the user from which the set of images was received. In some embodiments, this comprises providing information about the diagnosis back to the sample collection device for display to the user. In some embodiments, this comprises posting the diagnosis information to an account associated with the sample collection device. In some embodiments, if one or more specified conditions are detected in the sample based on the set of images, the set of images may be referred to a second user (e.g., a doctor or other specialist) for further analysis.

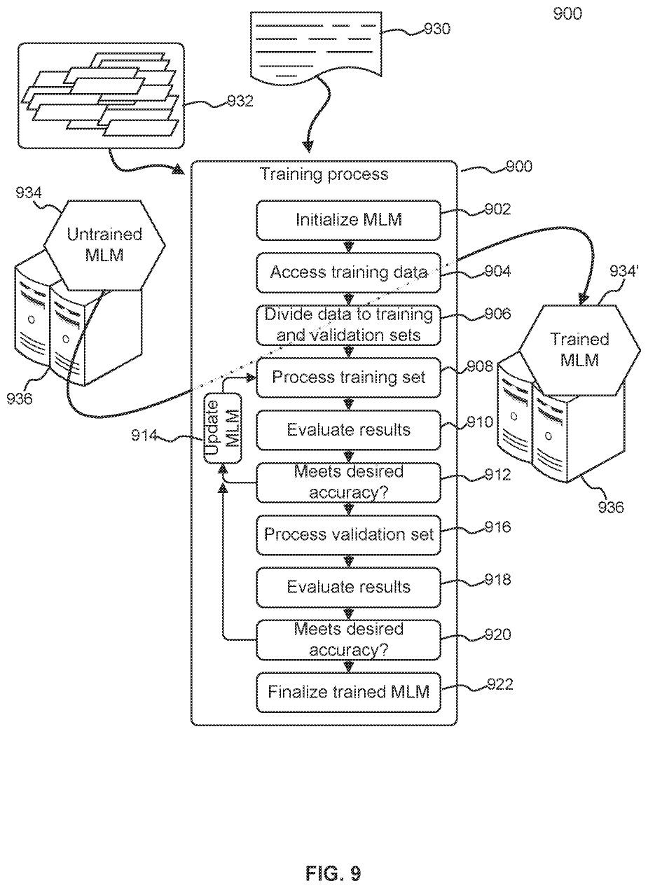

FIG. 9 illustrates an exemplary overall training process 900 of training a machine learning model to optimize collection of material sample data in accordance with aspects of the disclosed subject matter. Indeed, as shown in FIG. 9, the training process 900 is configured to train an untrained machine learning model 934 operating on a computer system 936 to transform the untrained machine learning model into a trained machine learning model 934′ that operates on the same or another computer system. In the course of training, as shown in the training process 900, at block 902, the untrained machine learning model 934 is optionally initialized with training features 930 comprising one or more of static values, dynamic values, and/or processing information.

At block 904 of training process 900, training data 932, is accessed, the training data corresponding to multiple items of input data. According to aspects of the disclosed subject matter, the training data is representative of a corpus of input data, (i.e., sensor, trigger, and/or media data) of which the resulting, trained machine learning model 934′ will receive as input. As those skilled in the art will appreciate, in various embodiments, the training data may be labeled training data, meaning that the actual results of processing of the data items of the labeled training data are known (i.e., the results of processing a particular input data item are already known/established). Of course, in various alternative embodiments, the corpus of training data 932 may comprise unlabeled training. Techniques for training a machine learning model with labeled and/or unlabeled data are known in the art.

With the training data 932 accessed, at block 906 the training data is divided into training and validation sets. Generally speaking, the items of input data in the training set are used to train the untrained machine learning model 934 and the items of input data in the validation set are used to validate the training of the machine learning model. As those skilled in the art will appreciate, and as described below in regard to much of the remainder of training process 900, in actual implementations there are numerous iterations of training and validation that occur during the overall training of the machine learning model.

At block 908 of the training process, the input data items of the training set are processed, often in an iterative manner. Processing the input data items of the training set includes capturing the processed results. After processing the items of the training set, at block 910, the aggregated results of processing the input data items of the training set are evaluated. As a result of the evaluation and at block 912, a determination is made as to whether a desired level of accuracy has been achieved. If the desired level of accuracy is not achieved, in block 914, aspects (including processing parameters, variables, hyperparameters, etc.) of the machine learning model are updated to guide the machine learning model to generate more accurate results. Thereafter, processing returns to block 902 and repeats the above-described training process utilizing the training data. Alternatively, if the desired level of accuracy is achieved, the training process 900 advances to block 916.

At block 916, and much like block 908, the input data items of the validation set are processed, and the results of processing the items of the validation set are captured and aggregated. At block 918, in regard to an evaluation of the aggregated results, a determination is made as to whether a desired accuracy level, in processing the validation set, has been achieved. At block 920, if the desired accuracy level is not achieved, in block 914, aspects of the in-training machine learning model are updated in an effort to guide the machine learning model to generate more accurate results, and processing returns to block 902. Alternatively, if the desired level of accuracy is achieved, the training process 900 advances to block 922.

At block 922, a finalized, trained machine learning model 934′ is generated. Typically, though not exclusively, as part of finalizing the now-trained machine learning model 934′, portions of the now-trained machine learning model that are included in the model during training for training purposes may be extracted, thereby generating a more efficient trained machine learning model 934′.

### CONCLUSION

Although the subject matter has been described in language specific to features and methodological acts, it is to be understood that the subject matter defined in the appended claims is not necessarily limited to the specific features or acts described herein. Rather, the specific features and acts are disclosed as exemplary forms of implementing the claims.

What is claimed is:

1. A computer-implemented method, comprising:
receiving a material sample at a sample collection device;
obtaining at least one initial image of the material sample via an image capture device of the sample collection device;
determining, based on an analysis of the at least one initial image via a trained machine learning model, a category for the material sample;
retrieving, based on the category for the material sample as determined via the trained machine learning model, a set of data collection instructions specific to the category of the material sample;
collecting, via the image capture device of the sample collection device, a set of images of the material sample in accordance with the set of data collection instructions; and
transmitting the set of images of the material sample as captured by the image capture device of the sample collection device to a service provider platform, the service provider platform including an additional trained machine learning model that correlates the set of images with a diagnosis of a condition associated with the material sample.

2. The computer-implemented method of claim 1, further comprising:
receiving information regarding the diagnosis of the condition that is associated with the material sample at the sample collection device; and
displaying, via the sample collection device, the information regarding the diagnosis of the condition.

3. The computer-implemented method of claim 1, further comprising posting the information regarding the diagnosis of the condition to an account of a user.

4. The computer-implemented method of claim 1, further comprising:
receiving, at the sample collection device, a collection cartridge that contains an additional material sample, the collection cartridge including a machine-readable code that indicates a particular type of the additional material sample in the collection cartridge;
retrieving, based on the particular type of the additional material sample indicated by the machine-readable code, an additional set of data collection instructions specific to the particular type of the additional material sample;
collecting, via the image capture device of the sample collection device, an additional set of images of the additional material sample in accordance with the additional set of data collection instructions; and
transmitting the additional set of images of the material sample as captured by the image capture device of the sample collection device to a service provider platform, the service provider platform including an additional trained machine learning model that correlates the additional set of images with an additional diagnosis of an additional condition associated with the material sample.

5. The computer-implemented method of claim 1, wherein the material sample comprises a biological sample collected from a user or a non-biological sample.

6. The computer-implemented method of claim 1, wherein the set of images are transmitted to the service provider platform via a wireless communication channel.

7. The computer-implemented method of claim 1, wherein the material sample is contained within a collection cartridge placed within the sample collection device.

8. The computer-implemented method of claim 7, wherein the collection cartridge is rotated, rocked, or vibrated to move the material sample into a testing compartment of the collection cartridge for imaging by the image capture device.

9. The computer-implemented method of claim 1, wherein the set of images include at least one of a bright field microscopy image of at least one portion of the material sample, a dark field microscopy image of the at least one portion of the material sample, or a fluorescence microscopy image of the at least one portion of the material sample.

10. The computer-implemented method of claim 1, wherein the data collection instructions direct a collection of at least one of one or more bright field microscopy images of at least one portion of the material sample, one or more dark field microscopy images of at least one portion of the material sample, or one or more fluorescence microscopy images of at least one portion of the material sample.

11. The computer-implemented method of claim 1, wherein the data collection instructions for collecting an image of the material sample include at least one of a resolution setting, a level of magnification setting, or a microscopy technique setting for the image of the material sample.

12. The computer-implemented method of claim 1, wherein the condition affects a user from which the material sample is collected, and wherein the diagnosis of the condition is included in anonymized data that is further analyzed by the service provider platform to detect a disease or sickness trends that affects a community.

13. A sample collection device, comprising:
one or more processors; and
memory including a plurality of computer-executable components that are executable by the one or more processors to perform a plurality of actions, the plurality of actions comprising:
receiving a material sample at the sample collection device;
obtaining at least one initial image of the material sample via an image capture device of the sample collection device;
determining, based on an analysis of the at least one initial image via a first trained machine learning model, a category for the material sample;
retrieving, based on the category for the material sample as determined via the first trained machine learning model, a set of data collection instructions specific to the category of the material sample;
collecting, via the image capture device of the sample collection device, a set of images of the material sample in accordance with the set of data collection instructions; and
correlating, via a second trained machine learning model, the set of images to a diagnosis of a condition associated with the material sample.

14. The sample collection device of claim 12, wherein the plurality of actions further comprise displaying, via the sample collection device, information regarding a diagnosis of the condition.

15. The sample collection device of claim 13, further comprising posting the information regarding the diagnosis of the condition to an account associated with a user.

16. The sample collection device of claim 12, wherein the material sample is contained within a collection cartridge placed within the sample collection device, and wherein the collection cartridge is rotated, rocked, or vibrated to move the material sample into a testing compartment of the collection cartridge for imaging by the image capture device.

17. The sample collection device of claim 12, wherein the set of images include at least one of a bright field microscopy image of at least one portion of the material sample, a dark field microscopy image of the at least one portion of the material sample, or a fluorescence microscopy image of the at least one portion of the material sample.

18. The sample collection device of claim 12, wherein the data collection instructions direct a collection of at least one of one or more bright field microscopy images of at least one portion of the material sample, one or more dark field microscopy images of at least one portion of the material sample, or one or more fluorescence microscopy images of at least one portion of the material sample.

19. The sample collection device of claim 12, wherein the data collection instructions for collecting an image of the material sample include at least one of a resolution setting, a level of magnification setting, or a microscopy technique setting for the image of the material sample.

20. One or more non-transitory computer-readable media of a sample collection device storing computer-executable instructions that upon execution cause one or more processors to perform acts comprising:
receiving, at the sample collection device, a collection cartridge that contains a material sample, the collection cartridge including a machine-readable code that indicates a particular type of the material sample in the collection cartridge;
retrieving, based on the particular type of the material sample indicated by the machine-readable code, a set of data collection instructions specific to the particular type of the material sample;
collecting, via an image capture device of the sample collection device, a set of images of the material sample in accordance with the set of data collection instructions; and
transmitting the set of images of the material sample as captured by the image capture device of the sample collection device to a service provider platform, the service provider platform including a trained machine learning model that correlates the set of images with a diagnosis of a condition associated with the material sample.
